(19) United States
(12) Patent Application Publication
(10) Pub. No.: US 2005/0272166 A1
(43) Pub. Date: Dec. 8, 2005

(54) Methods and systems for detection, identification and quantitation of macrolides and their impurities

(76) Inventors: Li Jin, Shoreline, WA (US); Joseph Therrien, Seattle, WA (US)

Correspondence Address: COZEN O' CONNOR, P.C., 1900 MARKET STREET, PHILADELPHIA, PA, 19103-3508, US

(21) Appl. No.: 11/122,533

(22) Filed: May 5, 2005

Related U.S. Application Data
(60) Provisional application No. 60/568,637, filed on May 6, 2004.

(51) Int. Cl. G01N 33/00

(52) U.S. Cl. 436/94

(57) ABSTRACT

The present invention relates to reverse-phase high performance liquid chromatography (RP-HPLC) methods and systems for detecting macrolides as well as detecting, identifying and quantifying impurities in samples containing a macrolide.

### CROSS REFERENCE TO RELATED APPLICATIONS

This application claims the benefit of U.S. Ser. No. 60/568,637, filed May 6, 2004, the disclosure of which is incorporated herein by reference in its entirety.

### FIELD OF THE INVENTION

The present invention relates to analytical methods and systems for detecting, identifying, and quantifying macrolides such as erythromycylamine and related compounds involving reverse-phase high performance liquid chromatography and electrochemical detection or mass spectrometry detection.

### BACKGROUND OF THE INVENTION

Macrolides describe a family of antibiotics used to treat a variety of bacterial infections. Macrolides are characterized chemically by a macrocyclic lactone ring structure of 14 to 16 atoms and usually at least one pendant sugar, amino sugar, or related moiety. Macrolides are believed to inhibit bacterial protein synthesis as a result of binding at two sites on the bacterial 50 S ribosome causing dissociation of transfer RNA and termination of peptide linking. Erythromycin, the first macrolide antibiotic, was discovered in 1952 and entered clinical use shortly thereafter. Erythromycin and the early derivatives (e.g., different salts and esters) are typically characterized by bacteriostatic or bactericidal activity for most gram positive bacteria, in particular streptococci, and good activity for respiratory pathogens. Macrolides proved to be safe and effective for many respiratory infections, and are useful in patients with penicillin allergy.

Macrolides typically have ultraviolet (UV) absorbance in the very low wavelength range (e.g., <220 nm), approaching the limits of photometric detection methods. The United States Pharmacopeia National Formulary (USP-NF) compendial assay method for Erythromycin (see structure below) involves RP-HPLC with L21 stationary phase (reverse-phase, rigid, spherical styrene-divinylbenzene copolymer, 5 to 10 μm particle diameter) using UV detection at 215 nm (see, e.g., pp 663-665 of USP-NF published Jan. 1, 2000). In fact, many reduced Erythromycin derivatives and related molecules such as 9-(S)-erythromycylamine (eryamine or PA2794; see structure below) have a UV maximum absorption band (UVₘₐₓ) well below 215 nm. For example, the UVₘₐₓ for 9-(S)-erythromycylamine occurs at about 191 nm, nearing the short wavelength limits of standard photometric detection methods. Accordingly, alternative detection methods such as electrochemical detection mass spectrometry detection methods have been found attractive (see, e.g., Whitaker, et al., J. Liq. Chromatogr. (1988), 11 (14), 3011-20; Pappa-Louisi, et al., J. Chromatogr., B: Biomed. Sci. Appl. (2001), 755 (1-2), 57-64; Kees, et al., J. Chromatogr., A (1998), 812 (1+2), 287-293; Hedenmo, et al., J. Chromatogr., A (1995), 692 (1+2), 161-6; Daszkowski, et al., J. Liq. Chromatogr. Relat. Technol. (1999), 22 (5), 641-657; and Dubois, et al., J. Chromatogr., B: Biomed. Sci. Appl. (2001), 753 (2), 189-202). These alternative methods, however, are principally designed for detection of macrolides in biological matrices such as blood plasma or other biological substance and are not optimized for separation of substantially pure macrolides (used as, e.g., active pharmaceutical ingredients (APIs)) from minor amounts of impurities, many of which are related macrolides with similar physical properties.

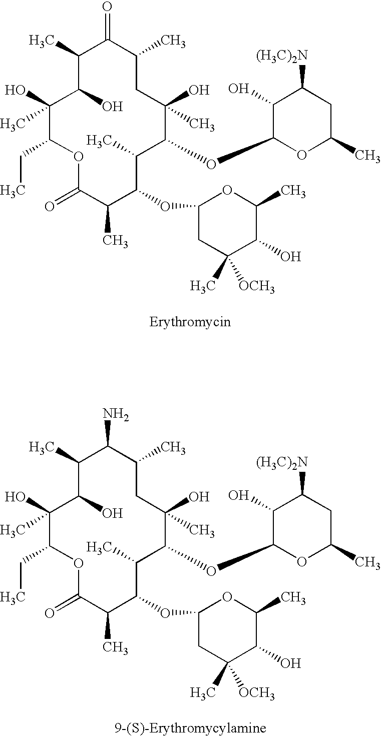

Because detection, identification, and quantitation of impurities in a macrolide sample are necessary for quality control, particularly when the macrolide is an API, there is a current need for assays that are suitably designed to detect, identify, and quantify macrolides, such as 9-(S)-erythromycylamine, and their impurities using HPLC-based assay methods. The methods and systems described herein help meet these and other needs.

### SUMMARY OF THE INVENTION

The present invention provides a method of detecting a macrolide in a test sample, wherein the major component of the test sample by weight is the macrolide, the method comprising:
a) applying the test sample on a reverse-phase high performance liquid chromatography (RP-HPLC) column;
b) eluting the test sample with a gradient mobile phase comprising a volatile buffer, water, acetonitrile, and alcohol; and
c) monitoring effluent from the column with an electrochemical detector or mass spectrometer detector to detect a current peak or mass peak, respectively, corresponding to the macrolide.

The present invention further provides a method of determining the purity of a test sample, wherein the major component of the test sample by weight is a macrolide, the method comprising:
a) applying the test sample on a reverse-phase high performance liquid chromatography (RP-HPLC) column;
b) eluting the sample with a gradient mobile phase comprising a volatile buffer, water, acetonitrile, and alcohol;
c) monitoring effluent from the column with an electrochemical detector to detect:
   i) a current peak corresponding to the macrolide; and
   ii) optionally one or more further current peaks corresponding to one or more impurities in the test sample; and
d) measuring one or more characteristics of the current peaks detected by the detector to calculate impurity content in the test sample.

The present invention further provides a method of identifying an impurity in a test sample, wherein the major component of the test sample by weight is a macrolide, the method comprising:
a) applying the test sample on a reverse-phase high performance liquid chromatography (RP-HPLC) column;
b) eluting the test sample with a gradient mobile phase comprising a volatile buffer, water, acetonitrile, and alcohol;
c) monitoring effluent from the column with a mass spectrometer detector to detect:
   i) a mass peak corresponding to the macrolide; and
   ii) a further mass peak corresponding to the impurity in the test sample; and
d) determining the mass of the further mass peak corresponding the impurity.

The present invention further provides a method of determining the amount of an impurity in a test sample, wherein the major component of the test sample by weight is a macrolide, the method comprising:
a) identifying the impurity by an HPLC-MS or HPLC-ECD assay;
b) determining the response factor for the impurity by the method comprising:
   i) running a known amount of the impurity and a known amount of the macrolide on a reverse-phase high performance liquid chromatography (RP-HPLC) column eluted with a mobile phase comprising an ion pair reagent, wherein the RP-HPLC column is outfitted with an ultraviolet (UV) detector having a detection wavelength between about 180 nm and about 220 nm;
   ii) monitoring column effluent with the UV detector to detect a first absorption peak at the detection wavelength, the first absorption peak corresponding to the impurity;
   iii) monitoring column effluent with the UV detector to detect a second absorption peak at the detection wavelength, the second absorption peak corresponding to the macrolide; and
   iv) calculating the response factor of the impurity using peak areas of the first and second absorption peaks; and
c) determining the amount of the impurity in the test sample by the method comprising:
   i) running the test sample under the same assay conditions of step b) to detect a third absorption peak corresponding to the impurity; and
   ii) calculating the amount of the impurity in the test sample using the response factor.

The present invention further provides a system for detecting impurities in a test sample of erythromycylamine, comprising:
a) a reverse-phase high performance liquid chromatography column comprising:
   i) a C18 column;
   ii) a gradient mobile phase comprising a mixture of eluent A and eluent B, the relative amounts of which vary during the course of elution, wherein eluent A consists essentially of about 60 to about 75 mM ammonium acetate in water and eluent B consists essentially of about 60 to about 75 mM ammonium acetate in a mixture of about 50 to about 70% by volume acetonitrile and about 30 to about 50% by volume methanol.
b) an electrochemical detector or mass spectrometer detector, wherein the electrochemical detector comprises a guard electrode, a screening electrode and a working electrode.

Example macrolides that can be detected by the methods and systems herein include, for example, 9-(S)-erythromycylamine, 9-(R)-erythromycylamine, erythromycin, erythromycin hydrazone, erythromycin, 9-imino erythromycin, erythromycin oxime, erythromycin B, erythromycin hydrazone B, 9-imino erythromycin B, erythromycylamine B, erythromycin hydrazone acetone adduct, 9-hydroxyimino erythromycin, erythromycylamine hydroxide, 9-hydroxyimino erythromycin B, erythromycylamine B hydroxide, erythromycylamine C, erythromycylamine D, azithromycin, clarithromycin, dirithromycin, roxithromycin, troleandomycin, derivatives thereof and the like.

The present invention further includes embodiments as provided in the Detailed Description.

### BRIEF DESCRIPTION OF THE DRAWINGS

FIGS. 1 and 2 show potential impurities of 9-(S)-erythromycylamine that can be detected, identified, and quantified according to the methods and systems of the invention.

FIG. 3 depicts an example synthesis of 9-(S)-erythromycylamine.

### DETAILED DESCRIPTION

The present invention provides, inter alia, HPLC-based methods and systems for detecting, identifying, and quantitating macrolides and their impurities using electrochemical (ECD) and/or mass spectroscopy (MS) as the detection method. The HPLC-ECD and HPLC-MS assays involve running a macrolide sample on a reverse-phase high performance liquid chromatography (RP-HPLC) column eluted with a gradient mobile phase containing a volatile buffer, water, acetonitrile, and alcohol. Effluent from the column is monitored with an electrochemical detector or mass spectrometer detector to detect a current peak or mass peak, respectively, that corresponds to the macrolide and/or potentially any impurities present in the sample. In some embodiments, the use of a mass spectrometer facilitates identification of compounds detected in the resulting chromatogram. Identified impurities in a macrolide sample can be further quantitated using an HPLC assay with photometric detection (e.g., HPLC-UV).

Macrolides according to the present invention include any of the known antibiotic or other macrolides and their derivatives. Typical macrolides are characterized by a 12-, 14-, or 16-membered macrocyclic lactone core structure. Macrolides are widely known in the art and are thoroughly described in, for example, Macrolide Antibiotics, ed. Satoshi Omura, Academic Press, Inc., Orlando, Fla., 1984, which is incorporated herein by reference in its entirety. In some embodiments, the macrolide has a relatively poor ultraviolet-visible (UV-VIS) absorption profile, for example, showing maximum absorption in the UV-VIS range (about 100 nm to about 900 nm) at a wavelength of about 180 nm to about 220 nm, about 180 nm to about 200 nm, or about 180 nm to about 195 nm. In further embodiments, the macrolide has a maximum absorption in the UV-VIS range at a wavelength of about 188, about 189, about 190, about 191, about 192, about 193, about 194, about 195, about 196, about 197, about 198, about 199, about 200, about 201, about 202, about 203, about 204, or about 205 nm.

Example macrolides that can be detected by the methods and systems herein include, for example, 9-(S)-erythromycylamine, 9-(R)-erythromycylamine, erythromycin, erythromycin hydrazone, erythromycin, 9-imino erythromycin, erythromycin oxime, erythromycin B, erythromycin hydrazone B, 9-imino erythromycin B, erythromycylamine B, erythromycin hydrazone acetone adduct, 9-hydroxyimino erythromycin, erythromycylamine hydroxide, 9-hydroxyimino erythromycin B, erythromycylamine B hydroxide, erythromycylamine C, erythromycylamine D, azithromycin, clarithromycin, dirithromycin, roxithromycin, troleandomycin, derivatives thereof and the like.

In some embodiments, the macrolide is 9-(S)-erythromycylamine.

In some embodiments, test samples suitable for analysis by the methods and systems of the present invention include at least one macrolide. In some embodiments, a test sample includes a macrolide which makes up the major component by weight in the test sample. The test sample can optionally include other minor amounts of components that can be referred to as impurities. In some embodiments, test samples are batches of substantially pure macrolide prepared by chemical synthetic procedures that often contain small amounts of impurities. As used herein, the term “impurities” refers to compounds other than the macrolide that is the subject of study. In some embodiments, one or more impurities can make up less than about 30%, less than about 20%, less than about 10%, less than about 5%, or less than about 1% by weight of the test sample.

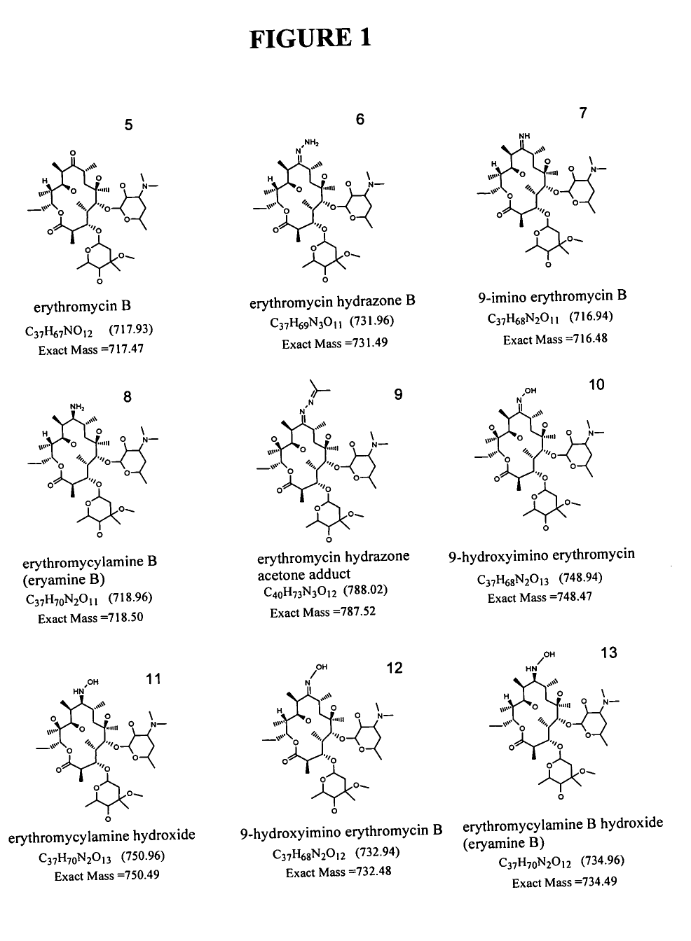

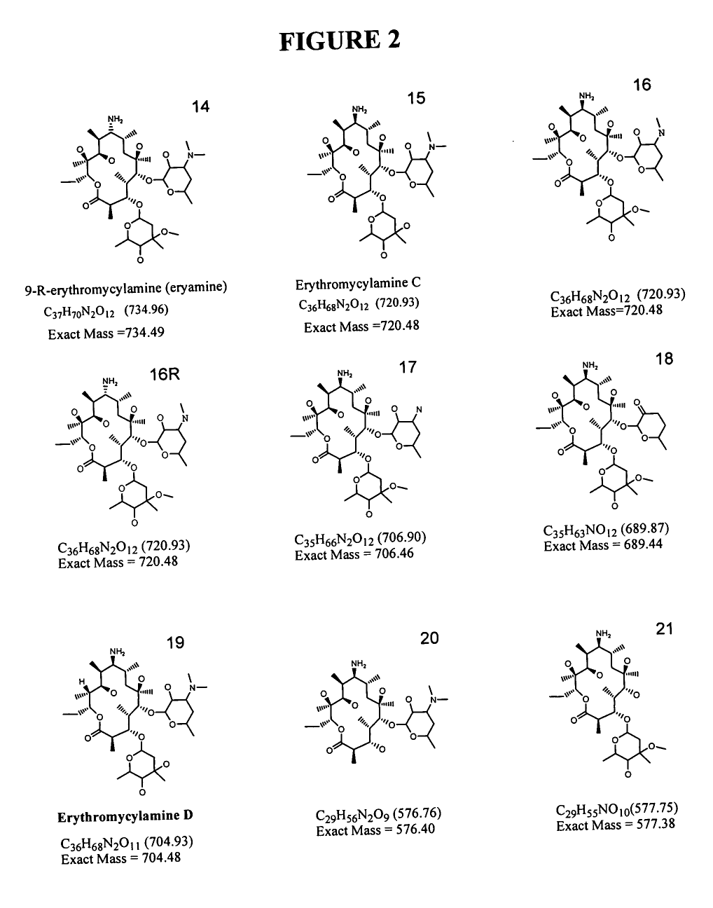

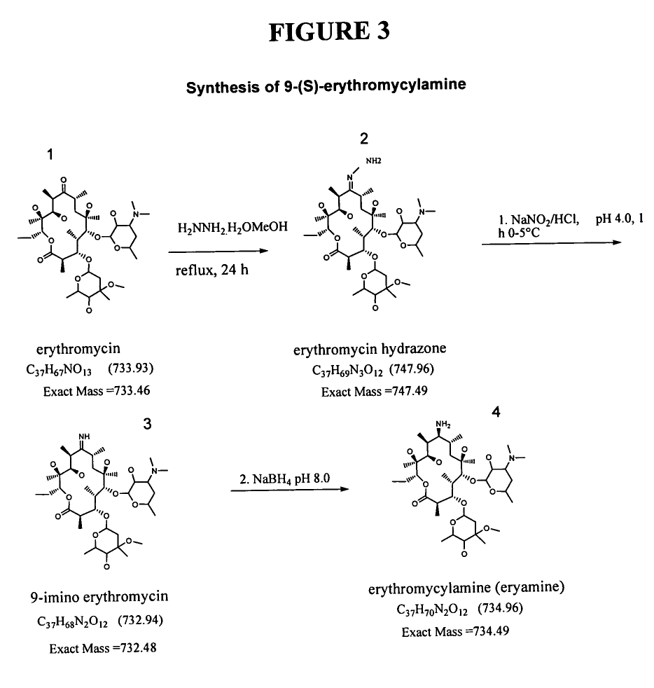

An impurity can often be another macrolide, such as a derivative of the macrolide making up the majority of the test sample. Impurities can be degradation products or carry-overs from chemical synthesis of the major macrolide component. In some embodiments, the impurities include one or more of the compounds shown in FIGS. 1 and 2, such as erythromycin B; erythromycin hydrazone B; 9-imino erythromycin B; erythromycylamine B; erythromycin hydrazone acetone adduct; 9-hydroxyimino erythromycin; erythromycylamine hydroxide; 9-hydroxyimino erythromycin B; erythromycylamine B hydroxide; 9-(R)-erythromycylamine; erythromycylamine C; erythromycylamine D; or a compound having Formula I, II, III, IV, or V:

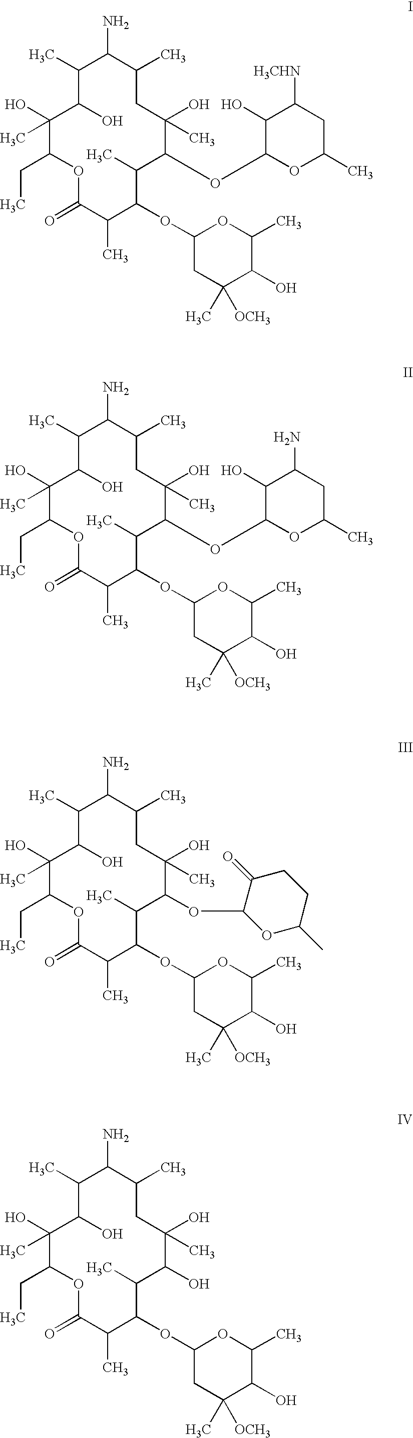

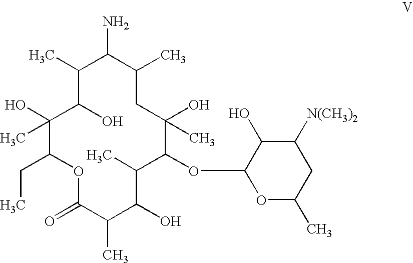

In some embodiments, an impurity is a compound of Formula VI, VII, VIII, IX, X, or XI:

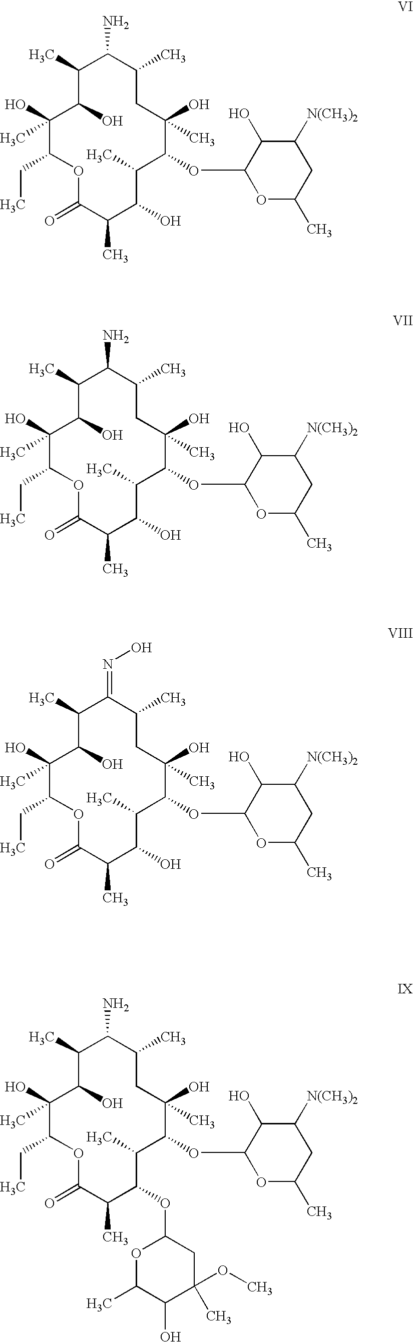

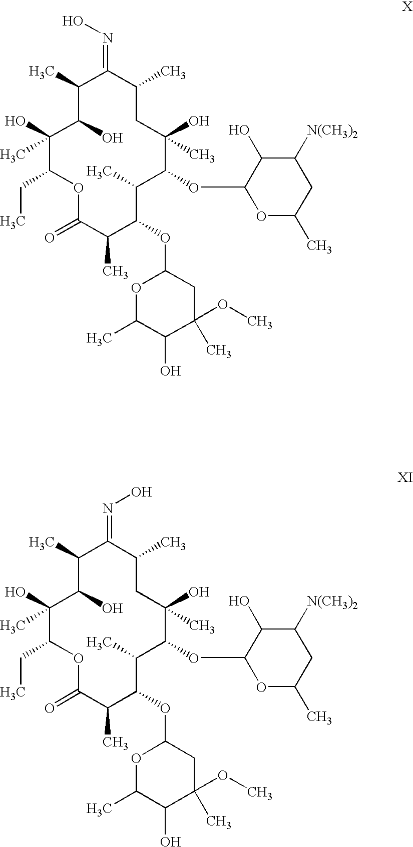

As used herein, the phrase “running a test sample” or the like in reference to an HPLC assay is meant to refer to the 1) application of a test sample to an HPLC column followed by 2) elution of the test sample with a mobile phase, where the resulting eluent is monitored with a detector capable of detecting a macrolide and/or impurities in the test sample.

HPLC-ECD and HPLC-MS Assays

The mobile phase for assays that are compatible with MS detection methods typically include volatile components. For example, mobile phase for HPLC-ECD and HPLC-MS assays according to the invention can contain a volatile buffer, water, acetonitrile, and alcohol.

Suitable volatile buffers include any buffering substance that maintains the mobile phase at the desired pH and does not interfere with detection of the macrolide by a mass spectrometer. In some embodiments, the volatile buffer comprises an ammonium salt such as ammonium acetate. The concentration of volatile buffer salt in the mobile phase can be about 40 to about 100, about 50 to about 80, or about 60 to about 75 mM. In some embodiments, the volatile buffer salt is present in the mobile phase at a concentration of about 67 mM. In further embodiments, the mobile phase has a pH of about 6 to about 8. In yet further embodiments, mobile phase has a pH of about 7.

Suitable organic components of the mobile phase include organic solvent, such as acetonitrile, and an organic modifier such as an alcohol to control peak shape and retention time. Example suitable alcohols include C₁-C₈ straight-chain and branched alcohols such as methanol, ethanol, isopropanol, and the like. In some embodiments, the alcohol is methanol. In some embodiments, the volume ratio of acetonitrile to alcohol in the mobile phase can be about 1:1 to about 2:1. In some embodiments, the volume ratio of acetonitrile to alcohol is about 3:2.

The mobile phase can be run through the HPLC column as a gradient elution. Accordingly, the mobile phase can be comprised of a mixture of two or more different eluent solutions, the proportions of which vary over the time course of the elution. In some embodiments, the mobile phase is comprised of a mixture of eluent A and eluent B, the relative amounts of which vary during the course of elution. In some embodiments, eluent A contains about 60 to about 75 mM ammonium acetate in water and eluent B contains about 60 to about 75 mM ammonium acetate in a mixture of about 50 to about 70 % by volume acetonitrile and about 30 to about 50% by volume methanol.

In further embodiments, eluent A contains about 65 to about 70 mM ammonium acetate in water and eluent B contains about 65 to about 70 mM ammonium acetate in a mixture of about 55 to about 60% by volume acetonitrile and about 40 to about 45% by volume methanol.

In yet further embodiments, eluent A contains about 67 mM ammonium acetate in water and eluent B contains about 67 mM ammonium acetate in a mixture of about 58% by volume acetonitrile and about 42% by volume methanol.

In yet further embodiments, the mobile phase can contain at any point in time of the elution a mixture of about 40 to about 75% by volume of eluent A and about 25 to about 60% by volume eluent B. In some embodiments, the proportion of eluent B is incrementally increased for a portion of time during elution.

The stationary phase can be composed of any reverse-phase solid support medium that in combination with the mobile phase allows for the detection of the macrolide and separation of the same from impurities. In some embodiments, the stationary phase contains a C8 to C18 matrix. In further embodiments, the stationary phase is a C18 matrix.

The test sample can be diluted with a sample diluent solution to form a diluted sample prior to introduction into the column. Suitable concentrations of macrolide in the diluted sample can be any suitable amount such as about 0.1 to about 5 mg/mL. In some embodiments, the concentration can be about 0.5 to about 1 mg/mL. Sample diluent can be the same or similar to the mobile phase. In some embodiments, the sample diluent is a mixture of water, acetonitrile and an alcohol such as methanol. In further embodiments, the sample diluent contains about 50 to about 90% water, about 10 to about 50% of a mixture of about 50 to about 70% acetonitrile and about 30 to about 50% methanol. In yet further embodiments, the sample diluent contains about 70% water and about 30% of a mixture of about 60% acetonitrile and about 40% methanol.

The electrochemical detector (ECD) can be any suitable detector capable of inducing and detecting oxidation or reduction of the macrolide. One example of a suitable ECD includes one that uses three electrodes: a guard electrode or cell, a screening electrode, and working electrode. The working electrode can be, for example, a platinum or glassy carbon electrode. Calibration of the electrodes can be carried out by any standard means known to the skilled artisan. In some embodiments, the ECD is set for detection of the macrolide and accompanying impurities by oxidation of the same. For example, the working electrode can be set to a potential suitable for oxidizing the macrolide, such as can be determined by any of various known methods such as cyclic voltammetry. Suitable potentials for the working electrode include greater than about 700 mV, greater than about 750 mV, and greater than about 800 mV. In some embodiments, the working electrode has a potential of about 800 to about 900 mV. In further embodiments, the working electrode has a potential of about 850 mV. Potentials for the guard electrode and reference electrode can be readily determined by the art skilled. For example, the guard electrode can have a potential of about 1000 mV and the reference electrode can have a potential less than that of the working electrode, such as from about −100 mV to about 100 mV. In some embodiments, the reference electrode has a potential of about 0 mV.

The mass spectrometer (MS) detector can include any MS detector capable of detecting and determining the mass/charge ratio of the macrolide. Suitable MS detectors are widely available, such as in connection with many commercial LC-MS instruments and their use in detecting organic compounds such as macrolides is routine in the art. In some embodiments, detection of the macrolide and any accompanying impurities can be carried out with the MS detector in positive mode. Ionization can be carried out by any suitable method, including electrospray or other means. Suitable MS parameters include a capillary temperature of about 150 to about 200° C. (e.g., about 180° C.) and a vaporizer set to about 300 to abut 400° C. (e.g., about 350° C.).

Elution of the macrolide according to the methods and systems of the invention can be carried out under a variety of temperatures and pressures, including ambient temperature and pressure. In some embodiments, elution is carried out at a constant temperature of about 10 to about 30, about 15 to about 25, or about 20° C. Temperature can be maintained below room temperature by outfitting the column with a chiller designed for such applications. Conversely, temperature can be maintained above room temperature by outfitting the column with a heater designed for such applications. Elution can also be carried out under air or an inert atmosphere.

Detection of the macrolide can be confirmed by comparing a chromatogram obtained according to the assay of the invention containing a peak believed to correspond to the macrolide with a chromatogram run under the same conditions showing a reference peak for a known sample of the macrolide. For example, a sample peak appearing within about 0.2 min of the reference peak can be considered confirmed. The amount of macrolide in a sample can also be quantified by comparing the area of a peak corresponding to the macrolide with the area of a peak in a chromatogram obtained for a reference sample (standard) containing a known amount of the macrolide.

The present invention further provides a method of determining the purity of a test sample. The method involves a) running the sample on a reverse-phase high performance liquid chromatography (RP-HPLC) column eluted with a gradient mobile phase comprising a volatile buffer, water, acetonitrile, and alcohol. Effluent is monitored with an electrochemical detector to detect: i) a current peak corresponding to the macrolide; and ii) optionally one or more further current peaks corresponding to one or more impurities in the sample (e.g., current peaks having a peak area of about 0.05% or more of the peak area due to the macrolide). Characteristics of the current peaks are then evaluated to calculate impurity content, for example, percentages of peak areas or peak height ratios can be used to assess and calculate impurity content (purity). In some embodiments, current peak area is determined for each detected impurity as well as the macrolide, and percent of total peak area for each is calculated.

An impurity in a sample can be identified by determining, for example, the mass of the peak corresponding to the impurity in a chromatogram obtained by an HPLC-MS method of the invention.

Quantitation of Macrolide Impurities with HPLC-UV

Impurities identified in test samples by the HPLC-MS and/or HPLC-ECD assays described hereinabove can be quantitated using an HPLC assay coupled with photometric detection (HPLC-UV assay).

Suitable mobile phase composition for the HPLC-UV assay can be any combination of liquid components that effectively elutes the desired macrolide, allows for separation of the macrolide from potential impurities, and allows photometric detection of the macrolide at the detection wavelength. In some embodiments, the mobile phase has negligible absorbance (e.g., measured with a spectrophotometer over a 1 cm pathlength) above about 205 nm. By “negligible” is meant absorbance of about 0.02 or less. In further embodiments, the mobile phase has an absorbance (e.g., measured with a spectrophotometer over a 1 cm pathlength) of less than about 0.5, less than about 0.3, or less than about 0.1 at the detection wavelength.

The mobile phase of the HPLC-UV assay can contain water, organic solvent, or a mixture thereof. Any suitable organic solvent that is miscible with water and does not interfere with detection of the macrolide at the detection wavelength can be used. In some embodiments, the organic solvent is acetonitrile. The mobile phase can contain 0 to 100% (v/v) water and 0 to 100% (v/v) organic solvent. In some embodiments, the mobile phase contains about 5 to about 75, about 10 to about 60, or about 20 to about 50% (v/v) organic solvent.

The mobile phase of the HPLC-UV assay can further include a buffer to stabilize the solution at a desired pH. Any buffer that does not interfere with the detection of the macrolide at the detection wavelength can be used. In some embodiments, the buffer is a phosphate or sulfate buffer. In further embodiments, the buffer is a sulfate buffer. Buffer concentration can be, for example, about 0.1 mM to about 1000 mM. In some embodiments, buffer concentration is about 1 mM to about 500 mM, about 5 mM to about 100 mM, or about 10 mM to about 30 mM. Any pH at which the macrolide is sufficiently stable such that it can be detected by the methods and systems of the invention is suitable. In some embodiments, the pH is about 1 to about 4. In further embodiments, the pH is about 3.

In some embodiments, the mobile phase includes an ion pair reagent, such as for example, a salt that facilitates retention of the macrolide on a reverse-phase column. Any ion pair reagent that is reasonably stable in the mobile phase solution, is capable of forming an ion pair with a charged form (e.g., protonated or deprotonated) of the macrolide, and does not interfere with elution or detection of the macrolide is suitable. Various suitable ion pair reagents are commercially available and HPLC techniques using the same are well known in the art. Some example ion pair reagents include alkylsulfonate salts such as (C₄-C₁₂ alkyl)sulfonate salts including sodium 1-octanesulfonate. The concentration of ion pair reagent in the mobile phase can be about 0.1 mM to about 1000 mM. In some further embodiments, ion pair concentration is about 1 mM to about 500 mM, about 5 mM to about 100 mM, or about 10 mM to about 30 mM. In some embodiments, the ion pair concentration is about 12 mM to about 15 mM.

The mobile phase can be run through the HPLC column as an isocratic elution or gradient elution. In embodiments where a gradient mobile phase is applied, the mobile phase can be comprised of a mixture of two or more different eluent solutions, the proportions of which vary over the time course of the elution. For example, the mobile phase can contain variable amounts of water, organic solvent, buffer, and ion pair reagent during elution. The variation in component amounts can be adjusted such that the gradient mobile phase maintains substantially constant absorbance at the detection wavelength during the course of elution. The variation in component amounts can also be adjusted to optimize peak shape, elution time, separation of macrolide from impurities, and other parameters.

In some embodiments, the mobile phase composition of the HPLC-UV assay is varied by eluting with one of or a mixture of two eluent solutions, each containing different amounts of water, organic solvent, buffer, and ion pair reagent. In some embodiments, a first eluent solution contains about 10 to about 30% (v/v) organic solvent, about 70 to about 90% (v/v) water, about 10 to about 20 mM ion pair reagent, and about 10 to about 15 mM buffer and a second eluent solution contains about 40 to about 60% (v/v) organic solvent, about 40 to about 60% (v/v) water, about 8 to about 15 mM ion pair reagent, and about 8 to about 12 mM buffer. In further embodiments, a first eluent solution contains about 20% (v/v) organic solvent, about 80% (v/v) water, about 15 mM ion pair reagent, and about 13 mM buffer and a second eluent solution contains about 50% (v/v) organic solvent, about 50% (v/v) water, about 12 mM ion pair reagent, and about 10.5 mM buffer. At any point during elution, the mobile phase can be composed of 100% of one of the two eluent solutions or a mixture of the two.

The stationary phase of the HPLC-UV assay can be composed of any reverse-phase solid support medium that in combination with the mobile phase allows for the detection of the macrolide and separation of the same from potential impurities. In some embodiments, the stationary phase contains a C8 to C18 matrix. In further embodiments, the stationary phase is a C18 matrix.

The sample can be diluted to form an diluted sample for introduction into the column. The diluted sample can have a macrolide concentration of about 1 to about 10 mg/mL. Sample diluent can be comprised of water buffered by Bis-Tris (e.g., about 20 to about 100 mM, about 30 to about 70 mM, or about 50 mM of Bis-Tris) and having a pH of about 6 to about 8, or about 7.

The UV detector monitoring effluent from the column can include any spectrophotometer capable of detecting absorption or transmission of UV wavelengths through a liquid sample. The detector can be tuned to a detection wavelength which can be constant for the duration of elution. In some embodiments, effluent is monitored at a wavelength of about 190 nm to about 210 nm, about 197 nm to about 205 nm, or about 200 nm. In some embodiments, the detection wavelength is about 200 nm.

The UV response factor (normalized peak area ratio of impurity to macrolide at a the detection wavelength) for each impurity identified by the HPLC-MS and/or HPLC-ECD assays described above can be determined by carrying out the above HPLC-UV assay on a reference sample containing a known amount of the impurity as well as a reference sample containing a known amount of the macrolide in high purity. For example, the response factor for an impurity can be calculated according to the following equations A, B and C:
Resp. Factor=(Normalized peak area of impurity)/(Normalized peak area of macrolide) (A)
where:
Normalized peak area of impurity=(Peak Area of impurity)×(% purity)/(concentration of impurity) (B)
Normalized peak area of macrolide=(Peak Area of macrolide)×(% purity)/(concentration of macrolide) (C)

Amount of impurity in a test sample containing an unknown amount of impurity can be determined by identifying the impurity using the HPLC-MS or HPLC-ECD assays described herein, followed by calculation of a response factor for the identified impurity by carrying out the above-described HPLC-UV assay on a reference sample of the identified impurity and reference sample of the macrolide, assaying a test sample according to the HPLC-UV assay described above and using the calculated response factor to calculate the amount of impurity in the test sample.

Systems

Also encompassed by the invention are systems including an assembly of the components described above. For example, a system of the invention can contain a) a reverse phase high performance liquid chromatography column (RP-HPLC) containing i) stationary phase comprising reverse phase solid support matrix; and ii) a mobile phase as described above; and b) an electrochemical or mass spectrometer detector.

Additional parameters for running and optimizing an HPLC assay according to the present invention are well within the knowledge of one skilled in the art as evidenced in the literature, for example, by Snyder et al., Practical HPLC Method Development, 2^{nd} ed., Wiley, New York, 1997, the disclosure of which is incorporated herein by reference in its entirety.

The invention is described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### EXAMPLES

### Example 1

HPLC Parameters for Electrochemical and Mass Spectrometer Detection of 9-(S)-Erythromycylamine

HPLC analyses of 9-(S)-erythromycylamine samples were carried out according to the parameters below. Percentages are by volume.

Instrumentation

Waters 2690 HPLC pump
ESA Model 5200A Coulochem II electrochemical detector
ESA Model 5010 analytical cell
ACE C18 HPLC column (150×4.6 mm, 3 μm, MAC-MOD, P/N#ACE-11-1546)
Finnigan SSQ7000 or JEOL LC-Mate Mass Spectrometer system
   Mobile Phase

Eluent A: 0.0671 M ammonium acetate in water vacuum filtered through 0.22 μm filter) at pH 7.04.

Eluent B: 0.0671 M ammonium acetate in 57.6% acetonitrile and 42.4% methanol by volume (vacuum filtered through 0.22 μm filter)

Sample Diluent: 71% C₁₈ disk polished Milli Q water and 29% mixture of 57.6% acetonitrile and 42.2% methanol.

Standard and Sample Preparation

9-(S)-Erythromycylamine standard solution and sample solution of 0.8 to 0.9 mg/mL were prepared in a 50.00 mL volumetric flask using sample diluent.

Column Parameters

Flow Rate: 1 mL/min
Column Temperature: 40° C.
Injection Volume for ECD: 10 μL
Injection Volume for MS: 50 μL

Run Time: 60 min

Gradient Table

| Time | % Eluent A | % Eluent B |
|---|---|---|
| 0 | 71 | 29 |
| 2 | 71 | 29 |
| 20 | 65 | 35 |
| 48 | 51 | 49 |
| 50 | 71 | 29 |
| 60 | 71 | 29 |

Electrochemical Detection Parameters
Guard Cell: 1000 mV
Electrode 1: 0 mV
Electrode 2: 850 mV
Noise Filter: 5 seconds
Range: 50 μA
   Mass Spectrometer Detection Parameters
APCI positive mode
Capillary Temperature: 180° C.
Vaporizer Temperature: 350° C.

### Example 2

HPLC Analyses of Different Lots of 9-(S)-Erythromycylamine

Samples of 9-(S)-erythromycylamine from different vendors were analyzed by HPLC using either or both mass spectrometer or electrochemical detection according to the parameters provided in Example 1. Tentative structure assignments were made based on mss, and corresponding structures are provided in FIGS. 1 and 2. The letters “ND” indicate “not detected.”

TABLE 2
HPLC-MS and HPLC-ECD Data for 9-(S)-Erythromycylamine
Lots 36188CA00 and 6E0101

| | | | | | | | | |
|---|---|---|---|---|---|---|---|---|
| | | | | | | | | MS |
| | | | | | | MS | MS | MW/EM of |
| Peak | ECD | ECD | ECD | MS | MS | Peak | Tentative | tentative |
| # | RT. | R. RT. | % Area | RT. | R. RT. | Mass | Structure | structure |

LOT # 36188CA00

| | | | | | | | | |
|---|---|---|---|---|---|---|---|---|
| 1 | 3.705 | 0.226246 | 0.14 | ND | — | — | — | — |
| 2 | 6.741 | 0.411639 | 0.32 | 7.05 | 0.419643 | 735.7 | 13 | 734.96/734.49 |
| 3 | 7.309 | 0.446324 | 0.83 | 7.69 | 0.457738 | 721.8 | 15, 16, 16R | 720.93/720.48 |
| 4 | 10.713 | 0.654189 | 0.43 | 11.02 | 0.655893 | 721.8 | 15, 16, 16R | 720.93/720.48 |
| 5 | 14.800 | 0.903762 | <0.01 | 14.80 | 0.880952 | 721.7 | 15, 16, 16R | 720.93/720.48 |
| 6 | 15.748 | 0.961651 | 0.30 | ND | — | — | — | — |
| 7 | 16.376 | 1 | 96.35 | 16.80 | 1 | 735.6, | 4 | 734.96/734.49 |
| | | | | | | 1470.3 | | |
| 8 | 26.410 | 1.612726 | 0.31 | 27.02 | 1.608333 | 749.8 | 10 | 748.94/748.47 |
| 9 | 30.653 | 1.871825 | 0.50 | 31.10 | 1.851190 | 719.8 | 8 | 718.96/718.50 |
| 10 | 33.139 | 2.023632 | 0.23 | ND | — | — | — | — |
| 11 | 36.646 | 2.237787 | 0.13 | ND | — | — | — | — |
| 12 | 37.289 | 2.277052 | 0.46 | 37.87 | 2.254167 | 736.7 | unknown | — |
| 13 | 39.376 | 2.404494 | 0.01 | 39.46 | 2.348810 | 759.8 | unknown | — |

LOT # 6E0101

| | | | | | | | | |
|---|---|---|---|---|---|---|---|---|
| 1 | 6.677 | 0.408379 | 0.27 | 7.17 | 0.419298 | 735.7 | 13 | 734.96/734.49 |
| 2 | 7.020 | 0.429358 | 0.14 | 7.44 | 0.435088 | 577.7 | 20 | 576.76/576.40 |
| 3 | 7.245 | 0.443119 | 0.61 | 7.81 | 0.456725 | 721.8 | 15, 16, 16R | 720.93/720.48 |
| 4 | 10.626 | 0.649908 | 0.08 | ND | — | — | — | — |
| 5 | 14.800 | 0.905199 | <0.01 | 15 | 0.877193 | 721.7 | 15, 16, 16R | 720.93/720.48 |
| 6 | 15.429 | 0.943670 | 0.03 | ND | | | | |
| 7 | 16.350 | 1 | 98.44 | 17.10 | 1 | 735.6, | 4 | 734.96/734.49 |
| | | | | | | 1470.3 | | |
| 8 | 26.275 | 1.607034 | 0.17 | ND | — | — | — | — |
| 9 | 32.731 | 2.001896 | 0.07 | ND | — | — | — | — |
| 10 | 36.519 | 2.233578 | 0.22 | 37.04 | 2.166082 | 749.6 | 10 | 748.94/748.47 |
| 11 | | | <0.01 | 39.57 | 2.314035 | 759.8 | unknown | — |

TABLE 3
HPLC-MS and Electrochemical Detector (ECD) Data for
9-(S)-Erythromycylamine Lots (S)AE/ii32/56 and 3535
Lot # (S)AE/ii32/56

| | | | | | | | | MS |
|---|---|---|---|---|---|---|---|---|
| | | | | | | MS | MS | MW/EM of |
| Peak | ECD | ECD | ECD | MS | MS | Peak | Tentative | tentative |
| # | RT. | R. RT. | % Area | RT. | R. RT. | Mass | Structure | structure |
| 1 | 3.672 | 0.226583 | 0.04 | ND | — | — | — | — |
| 2 | 6.675 | 0.411884 | 0.31 | 7.15 | 0.420588 | 735.7 | 13 | 734.96/734.49 |
| 3 | 7.236 | 0.446501 | 1.95 | 7.9 | 0.464706 | 721.7 | 15, 16, 16R | 720.93/720.48 |
| 4 | 10.605 | 0.654387 | 0.11 | ND | — | — | — | — |
| 5 | 14.091 | 0.869493 | 0.01 | 15.05 | 0.885294 | 721.7 | 15, 16, 16R | 720.93/720.48 |
| 6 | 15.388 | 0.949525 | 0.05 | ND | — | — | — | — |
| 7 | 16.206 | 1 | 97.14 | 17 | 1 | 735.6, | 4 | 734.96/734.49 |
| | | | | | | 1470.3 | | |
| 8 | 26.239 | 1.619092 | 0.13 | ND | — | — | — | — |
| 9 | 30.488 | 1.881279 | 0.12 | ND | — | — | — | — |
| 10 | 36.446 | 2.24892 | 0.11 | ND | — | — | — | — |
| 11 | 39.119 | 2.413859 | 0.04 | 39.55 | 2.326471 | 759.7 | unknown | — |

LOT # 3535

| | | | | | | | | MS |
|---|---|---|---|---|---|---|---|---|
| | | | | | | MS | MS | MW/EM of |
| Peak | ECD | ECD | ECD | MS | MS | Peak | Tentative | tentative |
| # | RT. | R. RT. | % Area | RT. | R. RT. | Mass | Structure** | structure |
| 1 | 3.666 | 0.22506 | 0.08 | 3.83 | 0.225294 | 751.8, | 11 | 750.96/750.49 |
| | | | | | | 809.8 | | |
| 2 | 6.674 | 0.409724 | 0.24 | 7.2 | 0.423529 | 735.7 | 13 | 734.96/734.49 |
| 3 | 7.241 | 0.444533 | 0.42 | 7.86 | 0.462353 | 721.7 | 15, 16, 16R | 720.93/720.48 |
| 4 | 10.606 | 0.651114 | 0.2 | 11.3 | 0.664706 | 721.7 | 15, 16, 16R | 720.93/720.48 |
| 5 | 15.411 | 0.946099 | 0.05 | 15.1 | 0.888235 | 721.7 | 15, 16, 16R | 720.93/720.48 |
| 6 | 16.289 | 1 | 77.81 | 17 | 1 | 735.6, | 4 | 734.96/734.49 |
| | | | | | | 1470.2 | | |
| 7 | 20.379 | 1.25109 | 0.03 | 19.84 | 1.167059 | 690.6 | 18 | 689.87/689.44 |
| 8 | 22.158 | 1.360304 | 0.09 | ND | — | — | — | — |
| 9 | 22.267 | 1.366996 | 0.08 | ND | — | — | — | — |
| 10 | 25.034 | 1.536865 | 0.04 | ND | — | — | — | — |
| 11 | 26.24 | 1.610903 | 0.09 | ND | — | — | — | — |
| 12 | 27.217 | 1.670882 | 0.44 | ND | — | — | — | — |
| 13 | 28.575 | 1.754251 | 0.49 | 28.33 | 1.666471 | 775.6 | unknown | — |
| 14 | 30.47 | 1.870588 | 0.27 | 29.66 | 1.744706 | 763.7 | unknown | — |
| 15 | 31.656 | 1.943397 | 0.05 | 31.25 | 1.838235 | 719.8 | 8 | 718.96/718.50 |
| 16 | 32.604 | 2.001596 | 2.86 | 32.68 | 1.922353 | 735.7 | unknown | — |
| 17 | 34.387 | 2.111057 | 0.01 | 33.4 | 1.964706 | 749.7 | 10 | 748.94/748.47 |
| 18 | 36.331 | 2.230401 | 16.37 | 35.55 | 2.091176 | 756.7 | unknown | — |
| 19 | 37.976 | 2.331389 | 0.08 | 36.9 | 2.170588 | 749.7 | 10 | 748.94/748.47 |
| 20 | 38.481 | 2.362392 | 0.07 | 39.11 | 2.300588 | 704.6 | 19 | 704.93/704.48 |
| 21 | 39.795 | 2.44306 | 0.09 | 39.58 | 2.328235 | 759.7 | unknown | — |
| 22 | 42.864 | 2.631469 | 0.06 | ND | — | — | — | — |
| 23 | 46.485 | 2.853766 | 0.12 | 47.38 | 2.787059 | 714.7 | unknown | — |

TABLE 4
HPLC-MS and Electrochemical Detector (ECD) Data for 9-(S)-Erythromycylamine
Lots 6E0201 and PDC325/Vd070202

| | | | | | | | | |
|---|---|---|---|---|---|---|---|---|
| | | | | | | | | MS |
| | | | | | | MS | MS | MW/EM of |
| Peak | ECD | ECD | ECD | MS | MS | Peak | Tentative | tentative |
| # | RT. | R. RT. | % Area | RT. | R. RT. | Mass | Structure | structure |

Lot # 6E0201

| | | | | | | | | |
|---|---|---|---|---|---|---|---|---|
| 1 | 3.572 | 0.22188 | 0.34 | 3.92 | 0.23772 | 751.6 | 11 | 750.96/750.49 |
| 2 | 6.503 | 0.40394 | 0.27 | 7.25 | 0.43966 | 735.7 | 13 | 734.96/734.49 |
| 3 | 6.768 | 0.4204 | 0.15 | 7.53 | 0.45664 | 577.7 | 20 | 576.76/576.40 |
| 4 | 7.256 | 0.45071 | 0.67 | 7.91 | 0.47968 | 721.8 | 15, 16, 16R | 720.93/720.48 |
| 5 | 10.478 | 0.65085 | 0.07 | 11.32 | 0.68648 | 721.8 | 15, 16, 16R | 720.93/720.48 |
| 6 | | | <0.03 | 14.12 | 0.85628 | 707.4 | 17 | 706.90/706.46 |
| 7 | 14.391 | 0.89391 | 0.09 | 14.98 | 0.90843 | 721.7 | 15, 16, 16R | 720.93/720.48 |
| 8 | 15.313 | 0.95118 | 0.03 | ND | ND | | | |
| 9 | 16.099 | 1 | 97.23 | 16.49 | 1 | 735.7 | 4 | 734.96/734.49 |
| 10 | | | <0.03 | 20.26 | 1.22862 | 690.6 | 18 | 689.87/689.44 |
| 11 | 20.433 | 1.26921 | 0.09 | 21.29 | 1.29109 | 735.7 | unknown | — |
| 12 | 23.053 | 1.43195 | 0.03 | 24.2 | 1.46756 | 735.6, | unknown | — |
| | | | | | | 777.4 | | |
| 13 | 26.309 | 1.63420 | 0.18 | 27.16 | 1.64706 | 749.8 | 10 | 748.94/748.47 |
| 14 | 30.596 | 1.90049 | 0.73 | 31.29 | 1.89751 | 719.8 | 8 | 718.96/718.50 |
| 15 | 32.770 | 2.03553 | 0.04 | ND | ND | — | — | — |
| 16 | 36.586 | 2.27256 | 0.07 | ND | ND | — | — | — |
| 17 | 38.816 | 2.41108 | 0.06 | ND | ND | — | — | — |

Lot # PDC325/Vd070202

| | | | | | | | | |
|---|---|---|---|---|---|---|---|---|
| 1 | 3.571 | 0.2219 | 0.12 | 3.92 | 0.23932 | 751.7, | 11 | 750.96/750.49 |
| | | | | | | 807.1 | | |
| 2 | 6.499 | 0.40384 | 0.33 | 7.23 | 0.44139 | 735.6 | 13 | 734.96/734.49 |
| 3 | 6.764 | 0.42031 | 0.18 | 7.52 | 0.4591 | 577.5 | 20 | 576.76/576.40 |
| 4 | 7.262 | 0.45125 | 0.25 | 7.92 | 0.48352 | 721.4 | 15, 16, 16R | 720.93/720.48 |
| 5 | 10.487 | 0.65165 | 0.03 | 11.29 | 0.68926 | 721.7 | 15, 16, 16R | 720.93/720.48 |
| 6 | ND | ND | — | 14.04 | 0.85714 | 707.5 | 17 | 706.90/706.46 |
| 7 | 14.401 | 0.89486 | 0.08 | 14.95 | 0.9127 | 721.6 | 15, 16, 16R | 720.93/720.48 |
| 8 | 15.319 | 0.9519 | 0.02 | ND | ND | — | — | — |
| 9 | 16.093 | 1 | 98.75 | 16.38 | 1 | 735.6 | 4 | 734.96/734.49 |
| 10 | — | — | <0.03 | 20.21 | 1.23382 | 690.6 | 18 | 689.87/689.44 |
| 11 | 26.331 | 1.63618 | 0.09 | 27.17 | 1.65873 | 749.7 | 10 | 748.94/748.47 |
| 12 | 30.678 | 1.90629 | 0.06 | 31.27 | 1.90904 | 719.5 | 8 | 718.96/718.50 |
| 13 | 38.839 | 2.41341 | 0.08 | 43.52 | 2.6569 | 716.5 | unknown | — |

TABLE 5
HPLC-MS Data for 9-(S)-Erythromycylamine
Lots PD2012/WRS/328/016 and 10006647

| | | | | | |
|---|---|---|---|---|---|
| | | | | | MS |
| | | | | MS | MW/EM of |
| Peak | MS | MS | MS | Tentative | tentative |
| # | RT. | R. RT. | Peak Mass | Structure | structure |

Lot # PD2012/WRS/328/016

| | | | | | |
|---|---|---|---|---|---|
| 1 | 7.353 | 0.4386 | 735.3 | 13 | 734.96/734.49 |
| 2 | 8.195 | 0.4888 | 721.5 | 15, 16, 16R | 720.93/720.48 |
| 3 | 15.546 | 0.9272 | 721.5 | 15, 16, 16R | 720.93/720.48 |
| 4 | 16.766 | 1.0000 | 735.5 | 4 | 734.96/734.49 |
| 5 | 22.489 | 1.3413 | 751.4 | unknown | — |
| 6 | 55.193 | 3.2920 | 690.5 | 18 | 689.87/689.44 |
| 7 | 55.723 | 3.3236 | 230.1/307.2/ | unknown | — |
| | | | 324.2/339.1 | | |

Lot # 10006647

| | | | | | |
|---|---|---|---|---|---|
| 1 | 7.367 | 0.4376 | 735.3 | 13 | 734.96/734.49 |
| 2 | 8.19 | 0.4865 | 721.5 | 15, 16, 16R | 720.93/720.48 |
| 3 | 14.47 | 0.8596 | 707.5 | 17 | 706.90/706.46 |
| 4 | 15.447 | 0.9176 | 721.5 | 15, 16, 16R | 720.93/720.48 |
| 5 | 16.834 | 1.0000 | 751.4 | 4 | 734.96/734.49 |
| 6 | 22.281 | 1.3236 | 751.5 | 11 (750.5) | |
| 7 | 31.757 | 1.8865 | 719.5 | unknown | — |
| 8 | 55.177 | 3.2777 | 690.5 | 18 | 689.87/689.44 |
| 9 | 55.73 | 3.3106 | 230.2/307.2/ | unknown | — |
| | | | 324.3/339.2 | | |

### Example 3

Identification and Quantitation of Impurities in a Sample of 9-(S)-Erythromycylamine

According to the procedure below, six macrolide impurities (see Table 6 and Formulas VI-XI above) were suspected as likely contaminants and confirmed as to their presence or absence in batches of 9-(S)-erythromycylamine for use as an API. Amounts of detected impurities were also quantitated.

TABLE 6

| Chemical Name | Formula | Possible Source |
|---|---|---|
| Decladinosyl-9-(R)-Erythromycylamine | VI | Synthetic Byproduct |
| Decladinosyl-9-(S)-Erythromycylamine | VII | Synthetic Byproduct |
| Decladinosyl-Erythromycylamine Oxime | VIII | Synthetic Byproduct |
| 9-(R)-Erythromycylamine | IX | Synthetic Byproduct |
| Erythromycin Oxime Base (Z-isomer) | X | Raw materials |
| Erythromycin Oxime Base (E-isomer) | XI | Synthetic Byproduct |

Reference samples of each of the above six suspected impurities as well as 9-(S)-erythromycylamine were purchased from Alembic, Inc. (India) and their structures were verified by FT-IR as well as HPLC-MS, which was carried out according to the procedure described in Example 1. FT-IR samples were prepared by mixing about 2 mg of reference sample with about 100 mg of dried KBr in an agate mortar and grinding into a fine powder. The powder was loaded into an 11 mm pellet die and compressed under vacuum. The IR spectrum was obtained by scanning 16 time as 4 cm⁻¹. IR spectra and MS data were consistent with each of the six compounds of Table 6.

Response factors for each of the above six suspected compounds were determined by the following procedure. Reference samples of each of the six suspected compounds were assayed by HPLC using photometric detection according to the HPLC-UV parameters provided below.

Instrumentation

The following equipment operated according the manufacturer's instructions was used for obtaining HPLC chromatograms. Equipment with comparable performance can be substituted.
Vacuum Degasser: Waters 2690
Pump: Waters 2690
Injector: Waters 2690
   50:50 mixture by volume of acetonitrile and water as needle wash
   100 μL injection loop
Pre-column: Phenomenex Security Guard with ODS cartridge (P/N AJO-4287)
Column: Phenomenex Column, C18(2), 150 mm×4.6 mm, 5 μm (P/N 00F-4252-E0)
   Column was installed in the direction of the eluent flow as instructed on the column label and placed in a Jones Chromatography column chiller/heater maintained at 20±1° C.
   Column was stored in 70:30 (v/v) acetonitrile:water when not in use.
Detector: Waters 2487 Dual Wavelength Detector
   Wavelength=200 nm
Column Chiller: Jones Chromatography model 7955
   Temperature was set to 20±1° C.
Data System: Perkin-Elmer Nelson Turbochrom data system, version 6.2.1
   Working Solutions
   Sample Diluent:
   50 mM Bis-Tris (Sigma)
   pH 7.4.
      Eluent A:
   20% acetonitrile (HPLC grade, Fisher)
   15 mM sodium 1-octanesulfonte (Fluka)
   13 mM Na₂SO₄ (Sigma)
   pH 3.1
      Eluent B:
   50% acetonitrile (HPLC grade, Fisher)
   12 mM sodium 1-octanesulfonte (Fluka)
   10.5 mM Na₂SO₄ (Sigma)
   pH 3.1
      HPLC Analysis

Sample analysis was carried out under the following parameters:
Flow rate: 1.0 mL/min
Injection Volume: 20 μL
Run Time: 40 min
Wavelength: 200 nm
Sample concentration: 0.5 mg/mL

The data system was set to acquire 1 point/second with a 40 min acquisition time.

A gradient mobile phase was applied according to Table A below.

TABLE A

| Time (min) | % Eluent A (v/v) | % Eluent B (v/v) |
|---|---|---|
| 0 | 70 | 30 |
| 1 | 70 | 30 |
| 20 | 30 | 70 |
| 26 | 30 | 70 |
| 27 | 0 | 100 |
| 31 | 0 | 100 |
| 33 | 70 | 30 |
| 40 | 70 | 30 |

The normalized peak area for each impurity reference sample and 9-(S)-erythromycylamine reference sample was calculated using equation (D) and the response factor was calculated using equation (E) where Area 1 is the peak area of a first injection and Area 2 is the peak area of a second injection.

$$\begin{matrix}\begin{matrix}Normalized peak area = ( Area 1 + Area 2 ) / 2 \times ( \% purity ) / \\ concentration ( mg \text{/} mL )\end{matrix} & ( D ) \\ Response factor = \frac{( Normalized peak area of impurity )}{( \begin{matrix}Normalized peak area of \\ 9 \text{-} ( S ) \text{-} erythromycylamine\end{matrix} )} & ( E )\end{matrix}$$

Calculated response factors for each of the 6 suspected impurities are provided in Table 7 below as well as for 9-(S)-erythromycylamine.

TABLE 7

| | Purity | Conc. | | Response |
|---|---|---|---|---|
| Chemical Name | (Area %) | mg/mL | Area | Factor |
| Decladinosyl-9-(R)- | 96.17 | 0.51 | 412.22 | 0.84 |
| Erythromycylamine (VI) | | | | |
| Decladinosyl-9-(S)- | 97.83 | 0.49 | 479.51 | 1.02 |
| Erythromycylamine (VII) | | | | |
| Decladinosyl- | 95.75 | 0.47 | 3070.61 | 6.87 |
| Erythromycylamine | | | | |
| Oxime (VIII) | | | | |
| 9-(R)-Erythromycylamine | 97.70 | 0.47 | 814.93 | 0.85 |
| (IX) | | | | |
| Erythromycin Oxime Base | 92.99 | 0.24 | 7402.75 | 7.74 |
| (Z-isomer) (X) | | | | |
| Erythromycin Oxime Base | 97.03 | 0.50 | 7370.83 | 7.70 |
| (E-isomer) (XI) | | | | |
| 9-(S)-Erythromycylamine | 99.18 | 0.55 | 956.84 | 1.00 |

Amounts of each of the six impurities in a test sample of 9-(S)-erythromycylamine was determined by running the test sample on the HPLC-UV column described above and using the response factors in Table 7. Results are provided in Table 8 below. No detectable amount of 9-(R)-erythromycylamine was observed.

TABLE 8

| | | | Conc. | % in |
|---|---|---|---|---|
| Chemical Name | Area | % Area | mg/mL | sample |
| Decladinosyl-9-(R)- | 7.9652 | 0.36 | 0.0105 | 0.43 |
| Erythromycylamine (VI) | | | | |
| Decladinosyl-9-(S)- | 2.6698 | 0.12 | 0.0029 | 0.12 |
| Erythromycylamine (VII) | | | | |
| Decladinosyl- | 10.9227 | 0.50 | 0.0018 | 0.50 |
| Erythromycylamine Oxime | | | | |
| (VIII) | | | | |
| 9-(R)-Erythromycylamine | NA | NA | NA | NA |
| (IX) | | | | |
| Erythromycin Oxime Base | 2.3329 | 0.11 | 0.0003 | 0.11 |
| (Z-isomer) (X) | | | | |
| Erythromycin Oxime Base | 21.9310 | 1.00 | 0.0032 | 1.00 |
| (E-isomer) (XI) | | | | |
| 9-(S)-Erythromycylamine | 2100.5869 | 95.51 | 2.3247 | 95.51 |

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable subcombination.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

1. A method of detecting a macrolide in a test sample, wherein the major component of said test sample by weight is said macrolide, said method comprising:
a) applying said test sample on a reverse-phase high performance liquid chromatography (RP-HPLC) column;
b) eluting said test sample with a gradient mobile phase comprising a volatile buffer, water, acetonitrile, and alcohol; and
c) monitoring effluent from said column with an electrochemical detector or mass spectrometer detector to detect a current peak or mass peak, respectively, corresponding to said macrolide.

2. The method of claim 1 wherein said macrolide is 9-(S)-erythromycylamine.

3. The method of claim 1 wherein said macrolide has maximum absorption in the ultraviolet-visible range at about 180 nm to about 220 nm.

4. The method of claim 1 wherein said volatile buffer is ammonium acetate.

5. The method of claim 1 wherein said mobile phase has a pH of about 6 to about 8.

6. The method of claim 1 wherein said alcohol comprises methanol.

7. The method of claim 1 wherein said mobile phase comprises a mixture of eluent A and eluent B, the relative amounts of which vary during the course of elution, wherein eluent A consists essentially of about 60 to about 75 mM ammonium acetate in water and eluent B consists essentially of about 60 to about 75 mM ammonium acetate in a mixture of about 50 to about 70% by volume acetonitrile and about 30 to about 50% by volume methanol.

8. The method of claim 1 further comprising quantifying the amount of said macrolide in said test sample by comparing the area or height of said current peak with a reference standard.

9. A method of determining the purity of a test sample, wherein the major component of said test sample by weight is a macrolide, said method comprising:
a) applying said test sample on a reverse-phase high performance liquid chromatography (RP-HPLC) column;
b) eluting said sample with a gradient mobile phase comprising a volatile buffer, water, acetonitrile, and alcohol;
c) monitoring effluent from said column with an electrochemical detector to detect:
i) a current peak corresponding to said macrolide; and
ii) optionally one or more further current peaks corresponding to one or more impurities in said test sample; and
d) measuring one or more characteristics of the current peaks detected by said detector to calculate impurity content in said test sample.

10. The method of claim 9 wherein said measuring is carried out by i) determining current peak area for each detected impurity and said macrolide; and ii) calculating the percentage of total current peak area due to said macrolide.

11. A method of identifying an impurity in a test sample, wherein the major component of said test sample by weight is a macrolide, said method comprising:
a) applying said test sample on a reverse-phase high performance liquid chromatography (RP-HPLC) column;
b) eluting said test sample with a gradient mobile phase comprising a volatile buffer, water, acetonitrile, and alcohol;
c) monitoring effluent from said column with a mass spectrometer detector to detect:
i) a mass peak corresponding to said macrolide; and
ii) a further mass peak corresponding to said impurity in said test sample; and
d) determining the mass of said further mass peak corresponding said impurity.

12. The method of claim 11 wherein said macrolide is 9-(S)-erythromycylamine.

13. The method of claim 11 wherein said impurity is a macrolide.

14. The method of claim 11 wherein said impurity is:
erythromycin B;
erythromycin hydrazone B;
9-imino erythromycin B;
erythromycylamine B;
erythromycin hydrazone acetone adduct;
9-hydroxyimino erythromycin;
erythromycylamine hydroxide;
9-hydroxyimino erythromycin B;
erythromycylamine B hydroxide;
9-(R)-erythromycylamine;
erythromycylamine C;
erythromycylamine D; or a compound having the Formula:

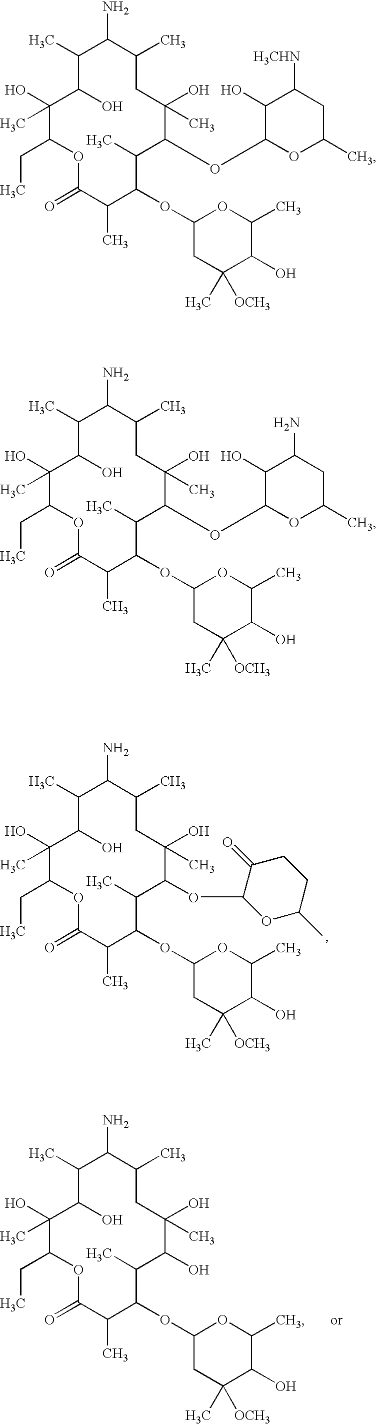

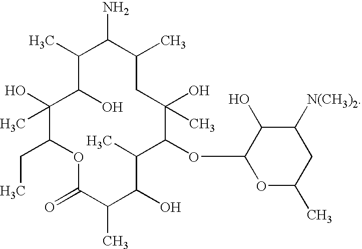

15. The method of claim 11 wherein said impurity is a compound of Formula VI, VII, VIII, IX, X, or XI:

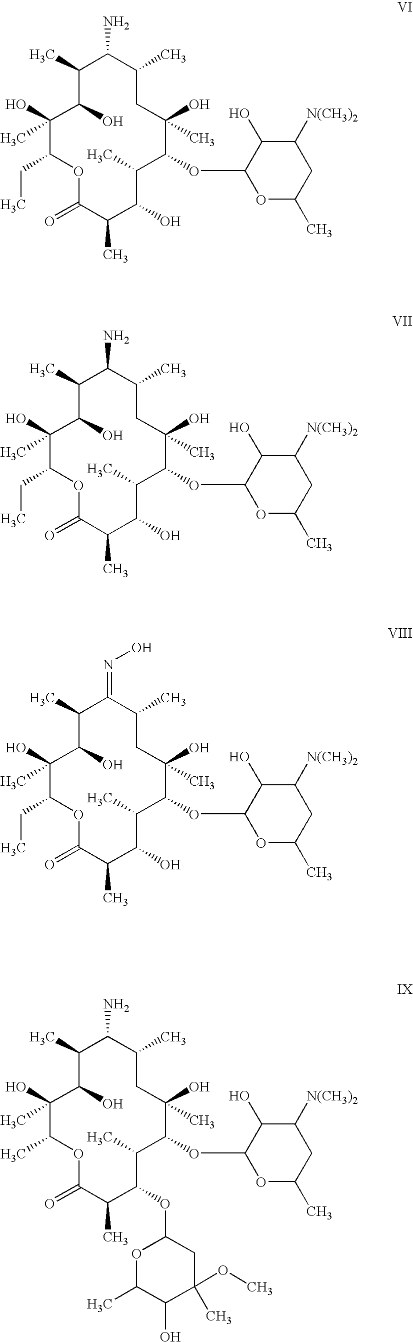

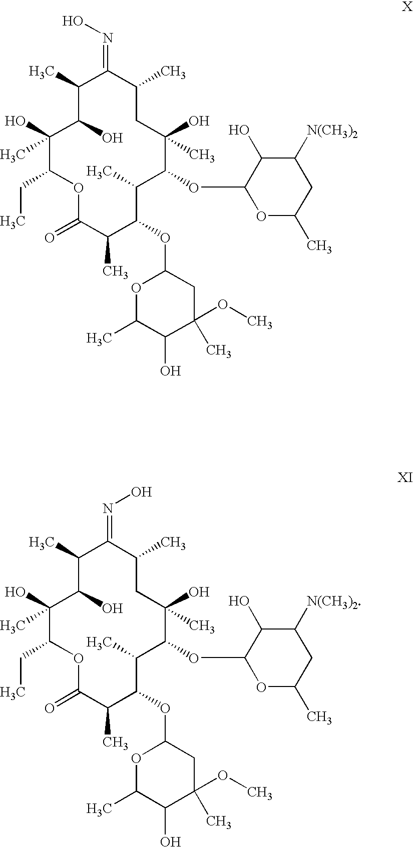

16. A method of determining the amount of an impurity in a test sample, wherein the major component of said test sample by weight is a macrolide, said method comprising:
a) identifying said impurity according to the method of claim 11;
b) determining the response factor for said impurity by the method comprising:
i) applying a known amount of said impurity and a known amount of said macrolide on a reverse-phase high performance liquid chromatography (RP-HPLC) column outfitted with an ultraviolet (UV) detector having a detection wavelength between about 180 nm and about 220 nm;
ii) eluting said known amount of said impurity with a mobile phase comprising an ion pair reagent;
iii) monitoring column effluent with said UV detector to detect a first absorption peak at said detection wavelength, said first absorption peak corresponding to said impurity;
iv) monitoring column effluent with said UV detector to detect a second absorption peak at said detection wavelength, said second absorption peak corresponding to said macrolide; and
v) calculating the response factor of said impurity using peak areas of said first and second absorption peaks; and
c) determining the amount of said impurity in said test sample by the method comprising:
i) running said test sample under the same assay conditions of step b) to detect a third absorption peak corresponding to said impurity; and
ii) calculating the amount of said impurity in said test sample using said response factor.

17. The method of claim 16 wherein said impurity is a compound of Formula VI, VII, VIII, IX, X, or XI:

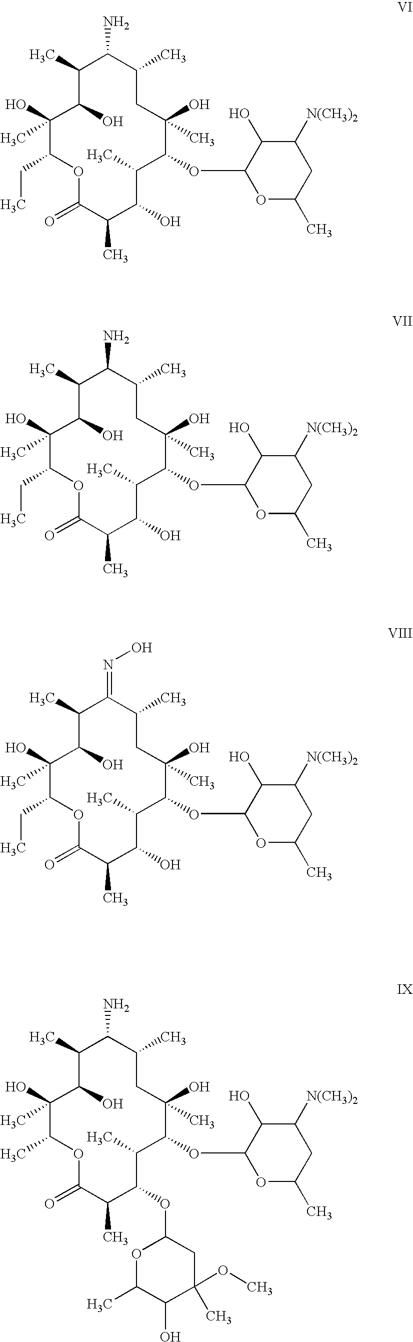

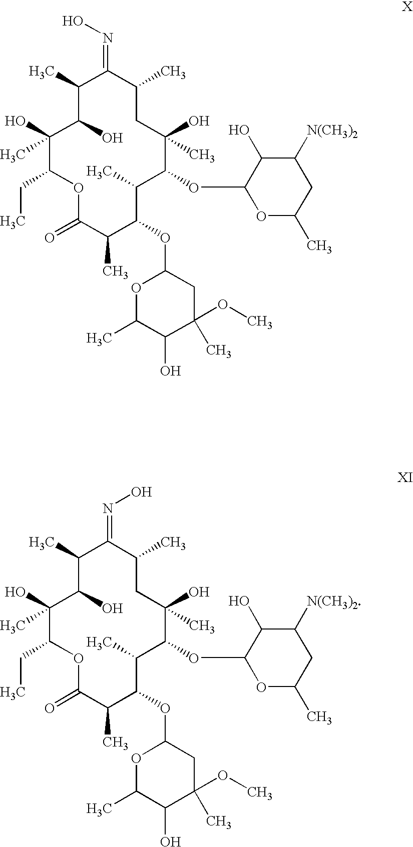

18. A system for detecting impurities in a test sample of 9-(S)-erythromycylamine, comprising:
a) a reverse-phase high performance liquid chromatography column comprising:
i) a C18 column;
ii) a gradient mobile phase comprising a mixture of eluent A and eluent B, the relative amounts of which vary during the course of elution, wherein eluent A consists essentially of about 60 to about 75 mM ammonium acetate in water and eluent B consists essentially of about 60 to about 75 mM ammonium acetate in a mixture of about 50 to about 70% by volume acetonitrile and about 30 to about 50% by volume methanol.
b) an electrochemical detector or mass spectrometer detector, wherein said electrochemical detector comprises a guard electrode, a screening electrode and a working electrode.
